(19) United States
(12) Patent Application Publication
(10) Pub. No.: US 2016/0271085 A1
(43) Pub. Date: Sep. 22, 2016

(54) TREATMENT OF AMYOTROPHIC LATERAL SCLEROSIS WITH LACTATE

(71) Applicant: Joel Steven Goldberg, Hillsborough, NC (US)

(72) Inventor: Joel Steven Goldberg, Hillsborough, NC (US)

(21) Appl. No.: 14/663,911

(22) Filed: Mar. 20, 2015

(51) Int. Cl. A61K 31/19

(52) U.S. Cl. CPC A61K 31/19

(57) ABSTRACT

Amyotrophic lateral sclerosis (ALS) is a devastating disease that leads initially to death of motor nerves with some progression to death of sensory and autonomic nerves. Experimental work shows that more electrical energy is required to successfully stimulate motor vs. sensory nerve components of a nerve and longer vs. shorter components of a nerve. This discovery explains sparing of the nerves of ocular motion and nerves of rectal and urethral sphincters in patients suffering from ALS and supports the energy hypothesis of ALS. Neurons and peripheral nerves are dependent upon glial cells and oligodendrocytes respectively to support their high energy demands. These supporting cells shuttle lactate to neurons and nerves. Lactate is required to sustain contraction of skeletal muscle. Administration of racemic or L-lactate in an amount greater than the capacity of the liver to oxidize will increase the available lactate to nerves and neurons and improve the symptoms of ALS.

### CROSS-REFERENCES TO RELATED APPLICATIONS

None

### FEDERALLY FUNDED RESEARCH

Not applicable

### BACKGROUND OF THE INVENTION

Amyotrophic lateral sclerosis (ALS) is a devastating disease that leads, initially to death of motor nerves with progression to death of sensory and autonomic nerves. There exist two forms of the disease 1) a hereditary or familial form that is associated with a defect in superoxide dismutase 1 (SOD1) enzyme and 2) a sporadic form that is not hereditary.

The etiology of ALS is unknown however evidence is emerging that there is an energy imbalance within nerve cells that produces deterioration of nerve cells when ATP is limited. (Martinez, 2012) This imbalance may be secondary to a defect in the transport of lactate from glial cells and oligodendrocytes to respective neurons and nerves.

Long motor neurons such as the sciatic nerve are often initially affected. Therefore foot drop is an early finding in patients with ALS. Short motor neurons such as cranial nerves III, IV, VI are often the last to be compromised in individuals with ALS. The nerves that control urethral and rectal voluntary sphincters are not usually affected.

Lactate has been shown by a number of investigators to be essential for the propagation of an action potential of a nerve as 1) a fuel source to produce ATP 2) a neurotransmitter or neuromodulator that acts as a catalyst to convert chemical to electrical energy and 3) as a buffer of hydrogen ions that is needed to sustain glycolysis. In 1932, Feng showed that sustained muscle contraction in injured sciatic nerves was enhanced by addition of lactate and that this response did not occur in an atmosphere of nitrogen suggesting that the effect was related to oxidation of lactate. (Feng, 1932)

Sodium lactate has been used in humans for experimental purposes. Infusions of sodium lactate have been used to initiate PTSD in sensitized individuals. (Jensen et al., 1997) Oral sodium lactate that has been ingested can significantly elevate serum lactate levels. (Crouse, Gerson, DeCarli, & Lieber, 1968; Lieber, Jones, Losowsky, & Davidson, 1962) Lactate in the blood is metabolized in the liver to bicarbonate that is subsequently metabolized to carbon dioxide and water. Lactate derived from sodium, potassium or calcium lactate is differentiated from lactate derived from lactic acid that is an end product of glycolysis. Sodium lactate is inexpensive and of low toxicity.

At the present time, there are no good therapies to treat the symptoms of ALS. Riluzole, the antiglutamate agent, appears to slow the progress of the disease. (Bensimon, Lacomblez, & Meininger, 1994)

In this invention, parenteral or oral administration of lactate distributes to the central and peripheral nervous systems providing nerves and neurons with a substrate that can be utilized to meet the energy demands of the motor nerves. In the familial form of ALS, lactate administration will improve motor function much less than in the sporadic form because the primary defect in the familial form is impaired oxidative phosphorylation within the mitochondria.

### DRAWINGS

None

### DETAILED DESCRIPTION OF THE INVENTION

Amyotrophic lateral sclerosis (ALS) is a degenerative disease that preferentially affects motor neurons. There is no cure for this disease and there is considerable morbidity associated with this illness.

In a human volunteer and using a standard nerve stimulator under ultrasound guidance it was discovered that less electric current was required to stimulate the sensory component of the median nerve compared to the motor component of the median nerve at the antecubital fossa. (Table 1) Furthermore it was discovered that stimulation of the median nerve at the wrist required less electrical current compared to stimulations at the antecubital fossa. Even though there could be some insulation effect from mantel fibers it was concluded from this experiment that for any given composite nerve, motor nerves require more energy for propagation of an effective action potential than sensory components. Also nerves of greater length require more energy to propagate an effective action potential than shorter nerves. This experiment provides an explanation of the phenotypic changes that occur in ALS. That is, motor nerves and those especially of the long corticospinal tract and sciatic nerve are most vulnerable to energy deficits compared to short motor nerves. Those cranial nerves that control ocular motor function (III, IV, and VI) are short and spared compared to other cranial nerves. Also sacral nerves with cell bodies in Onuf's nucleus are short. This discovery explains the deterioration in motor function in patients with ALS with sparing of the nerves of ocular motion and nerves that innervate the urethral and rectal sphincters. It also supports an energy hypothesis as a cause of ALS.

In addition to the energy required to propagate an action potential in the motor nerves, the energy required to release quanta of acetcylcholine within the motor endplate in larger muscles such as the biceps is greater than smaller muscles such as the muscles associated with ocular motion. Also the energy required to contract the urethral and rectal voluntary sphincters that are spared in ALS is less than an extremity muscle of similar size because the sphincters do not have reciprocal muscle antagonists to relax.

Evidence suggests that neurons and nerves in the central and peripheral nervous system are dependent upon lactate to support their high energy demands. Lactate becomes a substrate for neural tissue and is shuttled between the supporting glial cells and oligodendrocytes and the neurons or nerves respectively. Lactate can be transported to neurons and nerves via monocarboxylate transporters. Within neurons, and possibly nerves, lactate dehydrogenase 1 (LDH1) is the predominant form of LDH. LDH1 preferentially converts lactate to pyruvate that can then be oxidized to ATP through the Krebs cycle and oxidative phosphorylation. In patients who suffer from ALS the neurons and nerves that are deficient in lactate die because they are not able to provide the energy in the form of work needed for muscle contraction. Initially, recruitment of healthy motor nerves provide for muscle contraction but eventually these recruited nerves suffer the same fate as associated dying neurons and nerves. Subsequently, they also die because of lack of energy. (Lee et al., 2012; Martinez, 2012) ALS also affects sensory and autonomic nerves but the energy requirements to promote synaptic transmission in these nerves is considerably less than that associated with muscle contraction so the defect in lactate utilization is less apparent.

Infusions of lactate, both racemic and L-lactate, have been clinically trialed in patients with symptoms of posttraumatic stress disorder. (Martinez, 2012) Even though the liver removes approximately 70% of lactate in the circulation, it is possible to elevate blood lactate levels with oral or parenteral administration of sodium lactate. (Crouse, et al., 1968; Lieber, et al., 1962)

Experimental Section

In a human volunteer the left antecubital fossa and wrist were aseptically prepared. Under ultrasound guidance a nerve stimulation needle was positioned in the perineural tissue of the median nerve at the antecubital fossa and then at the wrist. The nerve was stimulated at 1 Hz with a Stimuplex Dig nerve stimulator (B. Braun Melsungen)

TABLE 1
Median nerve stimulation

| Location of median n. | Sensory | Motor |
|---|---|---|
| Antecubital | 1.05 ma | 1.71 ma |
| Wrist | 0.62 ma | 1.21 ma |

### REFERENCES

Bensimon, G., Lacomblez, L., & Meininger, V. (1994). A controlled trial of riluzole in amyotrophic lateral sclerosis. ALS/Riluzole Study Group. N Engl J Med, 330(9), 585-591.

Crouse, J. R., Gerson, C. D., DeCarli, L. M., & Lieber, C. S. (1968). Role of acetate in the reduction of plasma free fatty acids produced by ethanol in man. J Lipid Res, 9(4), 509-512.

Feng, T. P. (1932). The role of lactic acid in nerve activity. J Physiol, 76(4), 477-486.

Jensen, C. F., Keller, T. W., Peskind, E. R., McFall, M. E., Veith, R. C., Martin, D., et al. (1997). Behavioral and neuroendocrine responses to sodium lactate infusion in subjects with posttraumatic stress disorder. Am J Psychiatry, 154(2), 266-268.

Lee, Y., Morrison, B. M., Li, Y., Lengacher, S., Farah, M. H., Hoffman, P. N., et al. (2012). Oligodendroglia metabolically support axons and contribute to neurodegeneration. Nature, 487(7408), 443-448.

Lieber, C. S., Jones, D. P., Losowsky, M. S., & Davidson, C. S. (1962). Interrelation of uric acid and ethanol metabolism in man. J Clin Invest, 41, 1863-1870.

Martinez, B. A. (2012). Lactate-starved neurons in ALS. Dis Model Mech, 5(6), 711-712.

Having described my invention, I claim:

1. A method to treat the symptoms associated with amyotrophic lateral sclerosis (ALS) with parenteral or oral administration of L-lactate.

2. The method of claim 1 where the L-lactate is in the form of a salt.

3. The method of claim 1 where the L-lactate is derived from sodium lactate, potassium lactate or calcium lactate.
